(19) United States
(12) Patent Application Publication
(10) Pub. No.: US 2014/0357997 A1
(43) Pub. Date: Dec. 4, 2014

(54) INTRALUMINAL LEAD EXTRACTION WITH IMAGING

(71) Applicant: VOLCANO CORPORATION, San Diego, CA (US)

(72) Inventors: Jonathan Hartmann, San Diego, CA (US); Oren Levy, Emerald Hills, CA (US)

(73) Assignee: VOLCANO CORPORATION, San Diego, CA (US)

(21) Appl. No.: 14/290,379

(22) Filed: May 29, 2014

Related U.S. Application Data
(60) Provisional application No. 61/829,101, filed on May 30, 2013.

(51) Int. Cl. A61B 8/12; A61B 18/24; A61B 5/00

(52) U.S. Cl. CPC A61B 8/12; A61B 5/0095; A61B 18/24
USPC 600/439; 600/407

(57) ABSTRACT

This invention generally relates to intraluminal systems and devices for use in lead extraction procedures. According to certain aspects, an intraluminal system includes an cutting element configured to cut tissue coupled to an implantable device, and an imaging element configured to simultaneously image the tissue cutting. In certain embodiments, the cutting element is an ablation element.

### RELATED APPLICATION

The present invention claims the benefit of and priority to U.S. Provisional No. 61/829,101, filed May 30, 2013, which is incorporated by reference in its entirety.

### TECHNICAL FIELD

This invention generally relates to intraluminal system for lead extraction procedures.

### BACKGROUND

Cardiovascular implants, such as pacemakers and cardioverter defibrillators, play an important role in the treatment of heart disease. Pacemakers increase a patient's heart rate and slow heart rhythms by increasing the heart rate by harmonizing the heart's contraction. Implantable cardioverter defibrillators deliver an electric shock to stop abnormal, rapid heart rhythms. These cardiovascular implants are continually resorted to as both the patient population increases and the range of applications for treating patients with the devices increases.

Cardiovascular implants include a power generator (often referred to as the pacemaker or defibrillator itself) and cardiac leads, which are flexible wires coated with insulation that connect and deliver energy from the power generator to the heart. In pacemakers, the leads allow the implant to increase the heart rate by delivering bursts of electric energy to make the heart beat faster. In a defibrillator, a lead typically has coils configured to deliver high energy shock to the heart from an abnormal rhythm (often associated with ventricular tachycardia or fibrillation) to a more normal rhythm. The pulse generator includes electric circuits and a battery, usually placed under the skin on the chest wall beneath the collarbone. The leads run from the pulse generator through a vein, such as a vein under the collarbone, and are coupled directly to heart tissue in the heart. Typically, the leads are connected using one or more screws or hooks, and are attached directly to the right side of the heart. Once implanted, scar tissue forms around the lead in order to further secure the lead within the heart.

Occasionally, the leads to the cardiovascular implants must be removed or extracted. For example, implant and/or lead extraction may be necessary if the lead is no longer functioning properly. This can be due to a lead fracture, in which the lead no longer provides a reliable connection between the implantable device and the heart. In addition, leads often require removal when the tissue surrounding the lead becomes infected.

In order to remove a lead, it is necessary to detach the lead from the scar tissue formed around the lead in the heart. In addition, the lead may be attached along the length of a vein or vessel due to the formation of plaque around the lead. Specialized tools such as an excimer catheter are often required to remove the lead. Excimer catheters for lead removal include one or more ablative elements surrounding a lumen. Alternatively, catheters with mechanical cutting elements can be used. For removal, the excimer catheter is placed over a proximal end of the lead and into the lumen of the catheter. This allows the catheter to ride over the lead within the vessel towards the place where the lead is attached to the heart. While the catheter rides over the lead, the catheter ablates/cuts through scar tissue and plaque surrounding the lead so that the lead may be easily pulled from the vein.

Lead removal is a complex surgery that includes the inherent risk of tearing the veins containing the lead and/or perforating the heart tissue connected to the lead. As such, there is a need for improving current devices used to remove leads from the heart.

### SUMMARY

The invention recognizes that current lead extraction procedures are limited because prior art lead extraction devices do not allow visualization of the procedure within the lumen. Without visualization, the risk of perforating the heart coupled to or vessel surrounding the lead is increased. Devices and methods of the invention reduce risk associated with lead removal by providing systems that incorporate imaging with the cutting catheter, such as an excimer catheter. Such a system allowing an operator to visualize the lead while operating the catheter to remove the lead from the tissue. Systems of the invention may use ablative cutting elements, mechanical cutting elements or both as part of the cutting catheter.

In certain embodiments, the system is a combined cutting/imaging catheter, although the invention encompasses systems in which the cutting catheter is separate from the imaging element. The combined cutting/imaging catheter of the invention is able to ride over a cardiac lead and image the path of the catheter while the catheter removes any tissue/plaque bound to the sides of the cardiac lead and at the lead anchor site. By allowing a user to image visualize vessel/heart tissue before, during, and after the tissue excision, catheters of the invention are able to reduce risk associated with lead extraction procedures. The cutting/imaging catheter may include ablative cutting elements, mechanical cutting elements or both.

According to certain aspects, an intraluminal device of the invention includes a catheter body with an imaging element and a cutting element, such as a laser ablator or a mechanical cutter. The catheter body defines a lumen configured to receive an implantable device. The one or more cutting elements are configured to at least partially surround lumen at a distal end of the catheter and are at least partially disposed within the catheter body. Preferably, the cutting elements completely surround the lumen. Cutting elements of the invention are designed to remove tissue bound to a lead, such as using an ablation element to ablate tissue bound to the lead. This allows the lead to be more easily removed from the heart. The imaging element may be located on the body or as a separate component of the system. The imaging element allows a user to obtain real time images of the luminal surface during the extraction procedure.

Suitable tissue removal elements of the invention include, for example, ablation elements (such as optical fibers and electrodes) or mechanical cutting elements. In certain embodiments, the tissue removal elements are ablation elements, and they are in a concentric arrangement surrounding a lumen of the catheter. In this embodiment, the ablation elements may be arranged in a single row or more than one rows (e.g. 2, 3). In other embodiments, the ablation elements are in a random or densely-packed arrangement surrounding a lumen of the catheter. Preferably, ablation elements include an active end located on a distal end of the catheter body. In certain embodiments, the ablation elements are optical fibers configured to receive and emit laser energy from an active end of the optical fibers.

The systems of the invention also include one or more imaging elements. The imaging elements may be separate from the cutting catheter or located on the catheter body, thus providing an integrated device. Imaging elements of the invention may be a forward-looking imaging element, side-looking element, or combination of the two. Suitable imaging elements include, for example, ultrasound transducers and photoacoustic transducers. In one embodiment, the imaging element is placed on a distal end of the catheter body. In another embodiment, the imaging element is configured to surround a distal portion of the catheter body.

In another aspect of the invention, a catheter includes a body defining a first lumen and a second lumen. The catheter further includes a plurality of ablation elements at least partially disposed within the body and in an arrangement surrounding the first and second lumens. An inner member includes an imaging element and is moveably disposed within the second lumen of the body. The inner member can be deployed out of the second lumen to provide intraluminal images of the vessel and tissue during the lead extraction procedure.

### BRIEF DESCRIPTION OF THE DRAWINGS

FIG. 1 depicts generally illustrates an intraluminal device of the invention.

FIG. 2A depicts a catheter body with an imaging element and an ablation element according to certain embodiments.

FIGS. 2B and 2C illustrate the distal end of the intraluminal device shown in FIG. 2A according to certain embodiments.

FIG. 3A depicts another catheter body with an imaging element and an ablation element according to certain embodiments.

FIGS. 3B and 3C provide an image of the distal end of the intraluminal device shown in FIG. 3A according to certain embodiments.

FIG. 4A depicts another catheter body with an imaging element and an ablation element according to certain embodiments.

FIGS. 4B, 4C, and 4D provide an image of the distal end of the intraluminal device shown in FIG. 4A according to certain embodiments.

FIG. 5A depicts another catheter body with an imaging element and an ablation element according to certain embodiments.

FIGS. 5B and 5C provide an image of the distal end of the intraluminal device shown in FIG. 5A according to certain embodiments.

FIG. 6 illustrates a lead removal procedure using the intraluminal device of FIG. 2.

FIGS. 7-10 are isometric views showing different imaging planes generated by an imaging element.

FIGS. 11 and 12 depict an imaging element according to this embodiment that uses Fiber Bragg Gratings to generate acoustic energy.

### DETAILED DESCRIPTION

The invention is an intraluminal device (e.g. catheter) having both cutting elements (such as ablative elements) and imaging elements that facilitate visualization of the removal of leads associated with an implantable device. The visualization provided by the intraluminal catheter of the invention helps direct placement of the catheter within the vein and at the attachment site of the lead within the heart surface (or other luminal body surface). This advantageously assists in preventing inadvertent tearing of the vessel or perforation of a tissue surface while unbinding the lead from scar tissue and other plaque. The imaging element is located at or near the distal end of the intraluminal device to provide images at or near the ablation site. In certain embodiments, the imaging element is a forward looking imaging element. The imaging element may also be a side-viewing imaging element, or a combination of side viewing and forwarding imaging element. The imaging element is a part of an imaging assembly, such an IVUS or photoacoustic imaging assembly.

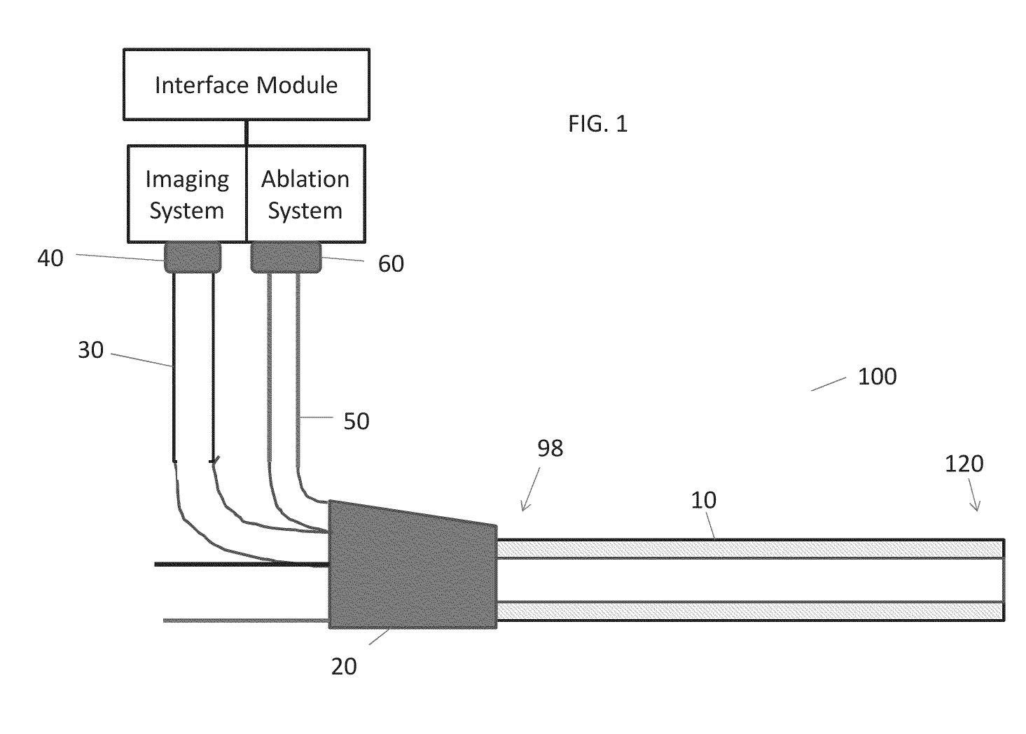

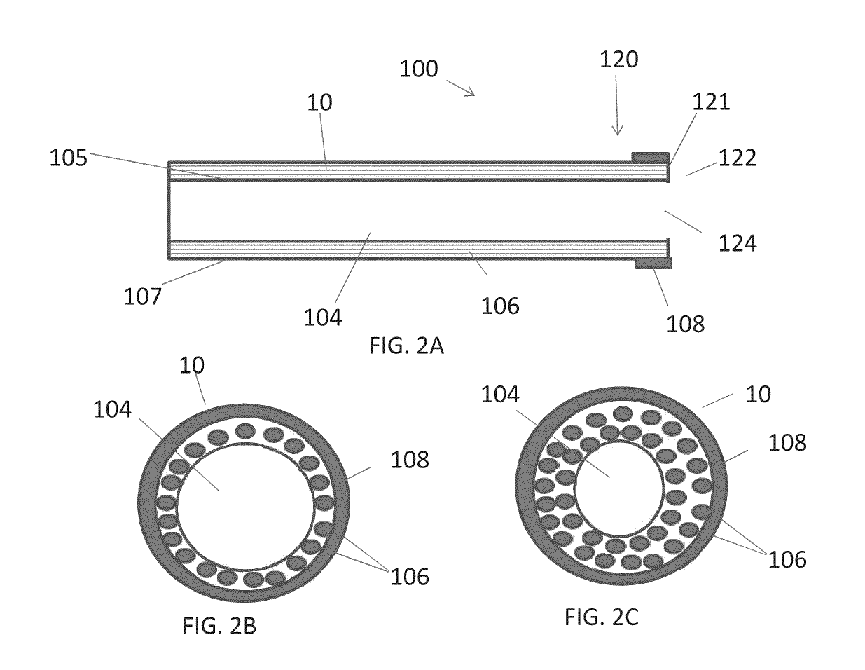

FIG. 1 depicts a schematic overview of an intraluminal device 100 according to certain embodiments. Generally, the intraluminal device includes a catheter body 10. The catheter body 10 includes at least one imaging element and at least one ablation element. As an alternative to an ablation system, the catheter may include a mechanical cutting element (such as a sharpened blade). The catheter body 10 includes a proximal portion 98 and a distal portion 120. The configuration of the imaging element and the ablation elements on the catheter body 10 are shown in more detail in FIGS. 2-5, which are described in more detail hereinafter. The catheter body 10 is coupled to a connector fitting 20, which is attached to a proximal portion 98 of the catheter body 10. The connector fitting 20 allows the signal lines to the ablation elements and the imaging elements to connect to their respective systems. The respective systems include an imaging system for the imaging element and an ablation system for the ablation element. The systems may be separate or may be formed in a single unit. In certain embodiments, the ablation system is a laser system, which is configured to provide laser energy to the ablation element. The imaging system is configured to send and receive imaging signals to and from the imaging element in order to obtain intraluminal images. As shown, the signal wires 30 (e.g. optical fibers, electrical wires, etc.) of the imaging element connect to the imaging system via an imaging connector 40. As shown, the signal wires 50 (e.g. optical fibers, electrical wires, etc.) of the ablation element connect to the ablation system via an ablation connector 60. The imaging system and the laser system may be connected to an interface module. The interface module for the imaging system may be the same or different from the interface module of the ablation system.

FIGS. 2A-5C depict various configurations of the imaging elements and cutting elements on the catheter body 10 of the invention. It is understood that the various configurations shown in FIGS. 2A-5C may be combined. For example, a catheter body 10 of the invention may include each of the imaging elements shown in FIGS. 2A-5C. As described in FIGS. 2A-5C, the catheters include an ablation element as a cutting element. However, the cutting element can also be a mechanical cutter such as a blade or serrated edge located on the distal end of the device.

FIG. 2A depicts a configuration of the catheter body 10 of the intraluminal device 100 according to certain embodiments. As shown in FIG. 2A, the catheter body 10 defines a center lumen 104. The center lumen 104 is sized to ride over an implantable device (e.g. a lead connected to a pacemaker or defibrillator). The center lumen 104 terminates at an opening 124 through which a lead may be loaded into the intraluminal device 100. The catheter body 10 has an outer surface 107 and an inner lumen surface 105. The catheter body 10 terminates a distal end 122. Although the distal end 122 is shown as a flat surface, the distal end 122 may be biased at an angle.

As further shown in FIG. 2A, one or more ablation elements 106 are at least partially disposed within the catheter body. The ablation elements 106 include an active end 121 located on the distal end 122 of the catheter body 110. The active end 121 is configured to release energy to ablate tissue in contact with or in close proximity to the distal end 122 of the catheter body 10. In certain embodiments, the ablation elements at least partially surround the center lumen 104. Preferably, the ablation elements 106 completely surround the center lumen. The ablation elements (e.g. optical fibers) may make up the inner surface of the catheter body, or an inner sheath may be placed between the ablation elements 106 and the center lumen 10. In certain embodiments, the one or more ablation elements 106 are optical fibers that run the length of the catheter body and terminate at the distal end 122. In this embodiment, the optical fibers are coupled to a laser system that provides laser energy through the optical fibers at the active end 121, which allows the optical fibers to ablate tissue. Alternatively, the active end 121 of the ablation elements 106 may be one or more electrodes coupled to the distal end 122. The electrodes would then be coupled to one or more signal lines that provide power to the electrodes. The ablation elements 106 are configured to ablation tissue, plaque, etc. bound to an electrode lead.

In addition, the intraluminal device 100 of FIG. 2A includes at least one imaging element 108. The imaging element is located at or near the distal end 122 of the catheter body 10. The imaging element may obtain images of a surface before, during, or after an ablation event or while the catheter body 10 is riding along a lead. The imaging element 108 may partially or completely surround the distal portion 120 of the catheter body 10. As shown, the imaging element 108 is surrounding the outer surface 107 of the catheter body 10. The imaging element 108 may include, for example, an ultrasound transducer or a photoacoustic transducer. The imaging element 108 may be a forward looking imaging element, a side looking imaging element, or a combined forward and side looking imaging element. Suitable imaging elements are described in more detail hereinafter.

FIGS. 2B and 2C provide a view of the distal end 122 of the catheter body 10 according to the embodiment depicted in FIG. 2A. As shown in both FIGS. 2C and 2C, the ablation elements 106 are disposed around the center lumen 104 in a concentric arrangement. As shown in FIG. 2B, the ablation elements 106 are shown arranged in a single row surrounding the center lumen 104. As shown in FIG. 2C, two rows of ablation elements 106 are provided. While the ablation elements 106 are shown in rows, the ablation elements may also be dispersed in a random or densely packed fashion. In addition, the row(s) of ablation elements 106 may be equally spaced apart. Any number of rows of ablation elements 106 may be used. However, the number of rows and/or number of ablation elements 106 may be constrained by vessel size requirements of the intraluminal device 100. As further shown in FIG. 2B and FIG. 2C, the imaging element 108 is surrounding the distal portion of the catheter body 10.

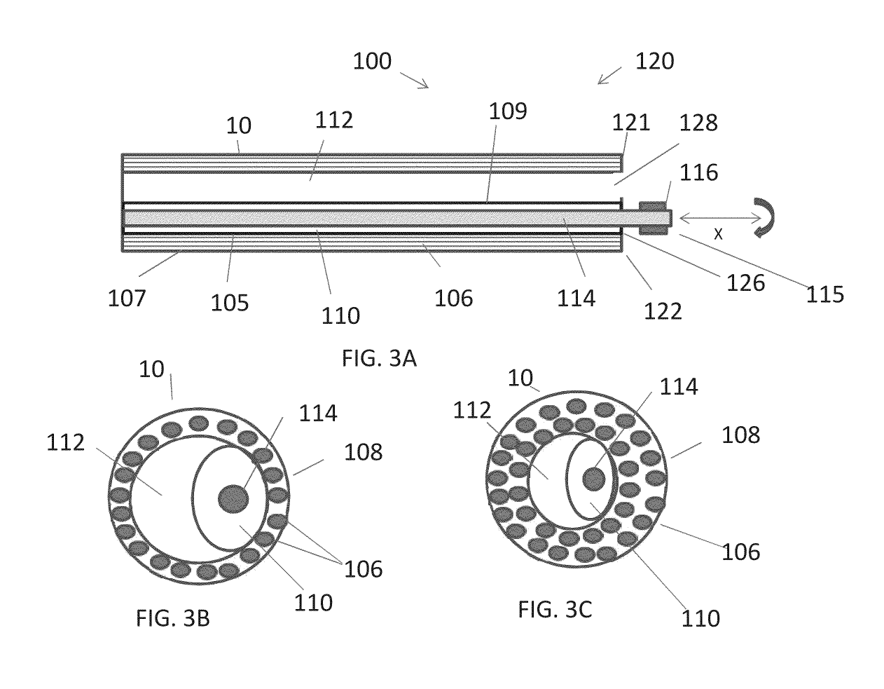

FIG. 3A depicts another configuration of the catheter body 10 of the intraluminal device 100 according to certain embodiments. As shown in FIG. 3A, the catheter body 10 defines a first lumen 112 and a second lumen 110. The first lumen 112 is sized to run over an implantable device (e.g. a lead connected to a pacemaker or defibrillator). The first lumen 112 terminates at an opening 128 through which a lead may be loaded into the intraluminal device 100. The catheter body 10 has an outer surface 107, an inner lumen surface 105, and a surface 109 separating the first lumen 112 and the second lumen 110. The catheter body 10 terminates a distal end 122. Although the distal end 122 is shown as a flat surface, the distal end 122 may be biased at an angle.

As further shown in FIG. 3A, the one or more ablation elements 106 are at least partially disposed within the catheter body 10. In certain embodiments, the ablation elements 106 at least partially surround the first lumen 112 and the second lumen 110. Preferably, the ablation elements 106 completely surround the lumens 112 and 110. The ablation elements (e.g. optical fibers) may make up the inner surface 105 of the catheter body, or an inner sheath may be placed between the ablation elements 106 and the first and second lumens 110, 112. A surface 109 separates the first and second lumens 112, 110.

The ablation elements 106 include an active end 121 at the distal end 122 of the catheter body 110. The active end 121 is configured to release energy to ablate tissue in contact with or in close proximity to the distal end 122 of the catheter body 10. In certain embodiments, the one or more ablation elements 106 are optical fibers that run the length of the catheter body and terminate at the distal end 122. In this embodiment, the optical fibers are coupled to a laser system that provides laser energy through the optical fibers at the active end 121, which allows the optical fibers to ablate tissue. Alternatively, the active end 121 of the ablation elements 106 may be one or more electrodes coupled to the distal end 122. The electrodes would then be coupled to one or more signal lines that provide power to the electrodes. The ablation elements 106 are configured to ablation tissue, plaque, etc. bound to electrode lead.

In addition, the intraluminal device 100 as shown in FIG. 3A includes an inner member 114. The inner member 114 is moveably disposed within the second lumen 110. The inner member 114 move longitudinally (as indicated by arrow x) forward and backwards within the second lumen 110. In addition, the inner member can be configured to rotate. The inner member 114 includes an imaging element 116 located on a distal portion 115 of the inner lumen 114. The imaging element 116 is configured to take images of a tissue surface when the inner member 114 is deployed out of the opening 126 of the second lumen 124. The inner member 114 may be deployed before, during, and after an ablation event or during movement of the catheter body 10 along a lead in order to obtain images of the luminal surface. The imaging element 116 may partially or completely surround the distal portion 115 of the inner member 114. Alternatively or additionally, the imaging element 116 may be placed on a distal end of the inner member 114. The imaging element 116 may include, for example, an ultrasound transducer, optical coherence tomography element, or a photoacoustic transducer. The imaging element 116 may be a forward looking imaging element, a side looking imaging element, or a combined forward and side looking imaging element. Suitable imaging elements are described in more detail hereinafter.

FIGS. 3B and 3C provide a view of the distal end 122 of the catheter body 10 according to the embodiment depicted in FIG. 3A. As shown in both FIGS. 3B and 3C, the ablation elements are disposed around the first lumen 112 and the second lumen 110 in a concentric arrangement. As shown in FIG. 3B, the ablation elements 106 are shown arranged in a single row surrounding the center lumen 104. As shown in FIG. 3C, two rows of ablation elements 106 are provided. While the ablation elements 106 are shown in rows, the ablation elements may also be dispersed in a random or densely packed fashion. In addition, the row(s) of ablation elements 106 may be equally spaced apart. Any number of rows of ablation elements 106 may be used. However, the number of rows and/or number of ablation elements 106 may be constrained by vessel size requirements of the intraluminal device 100. As further shown in FIG. 3B and FIG. 3C, the inner member 114 is disposed within the second lumen 112 of the catheter body 10.

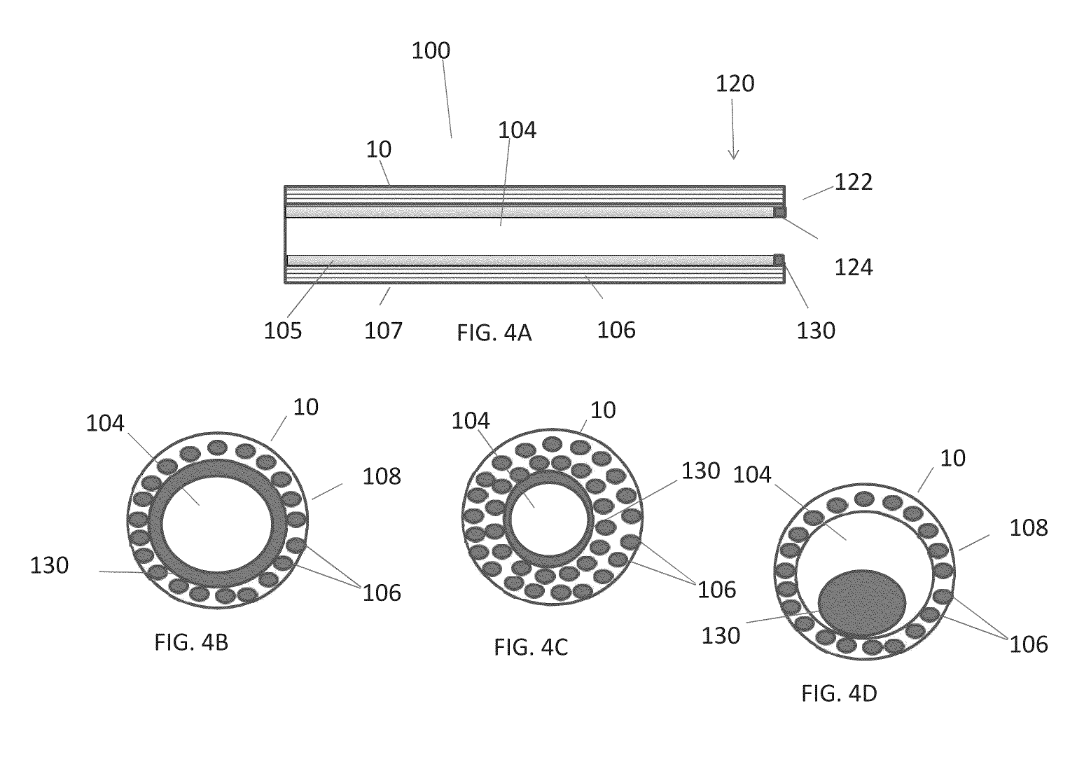

FIG. 4A depicts a configuration of the catheter body 10 of the intraluminal device 100 according to certain embodiments. As shown in FIG. 4A, the catheter body 10 defines a center lumen 104. The center lumen 104 is sized to run over an implantable device (e.g. a lead connected to a pacemaker or defibrillator). The center lumen 104 terminates at an opening 124 through which a lead may be loaded into the intraluminal device 100. The catheter body 10 has an outer surface 107 and an inner lumen surface 105. The catheter body 10 terminates a distal end 122. Although the distal end 122 is shown as a flat surface, the distal end 122 may be biased at an angle.

As further shown in FIG. 4A, one or more ablation elements 106 are at least partially disposed within the catheter body 10. The ablation elements 106 include an active end 121 located at the distal end 122 of the catheter body 110. The active end 121 is configured to release energy to ablate tissue in contact with or in close proximity to the distal end 122 of the catheter body 10. In certain embodiments, the ablation elements at least partially surround the center lumen 104. Preferably, the ablation elements 106 completely surround the center lumen 104. In certain embodiments, the one or more ablation elements 106 are optical fibers that run the length of the catheter body from the proximal end and to the distal end 122. In this embodiment, the optical fibers are coupled to a laser system that provides laser energy through the optical fibers at the active end 121, which allows the optical fibers to ablate tissue. Alternatively, the active end 121 of the ablation elements 106 may be one or more electrodes coupled to the distal end 122. The electrodes would then be coupled to one or more signal lines that provide power to the electrodes. The ablation elements 106 are configured to ablation tissue, plaque, etc. bound to electrode lead.

In addition, the intraluminal device 100 of FIG. 4A includes at least one imaging element 130 located on the distal end 122 of the catheter body 10. The imaging element 130 may be placed anywhere on the distal end 122 of the catheter body 10. The placement of the imaging element 130 on the distal end 122 is ideal for forward looking imaging because the imaging element is positioned to send and receive signals in front of the catheter body 10. As shown, the imaging element 130 is placed in a concentric ring on the distal end 122 of the catheter body 120, which surrounds the center lumen 104. For example, the imaging element may be a plurality of ultrasound transducer arranged on the distal end of the catheter body. In certain embodiments and as shown, the imaging element 130 is placed on the inner lumen surface 105. The imaging element may obtain images of a surface before, during, or after an ablation event or while the catheter body 10 is riding along a lead. The imaging element 130 may include, for example, an ultrasound transducer or a photoacoustic transducer. Suitable imaging elements are described in more detail hereinafter.

FIGS. 4B, 4C, and 4D provide a view of the distal end 122 of the catheter body 10 according to the embodiment depicted in FIG. 4A. As shown in both FIGS. 4B and 4C, the ablation elements are disposed around the center lumen 104 in a concentric arrangement. As shown in FIG. 4B, the ablation elements 106 are shown arranged in a single row surrounding the center lumen 104. As shown in FIG. 4C, two rows of ablation elements 106 are provided. While the ablation elements 106 are shown in rows, the ablation elements 106 may also be dispersed in a random or densely packed fashion. In addition, the row(s) of ablation elements 106 may be equally spaced apart. Any number of rows of ablation elements 106 may be used. However, the number of rows and/or number of ablation elements 106 may be constrained by vessel size requirements of the intraluminal device 100. As further shown in FIG. 4B and FIG. 4C, the imaging element 130 is placed on the distal end 122 of the catheter body 10, and the imaging element 130 surrounds the center lumen 104. FIG. 4D depicts an alternative embodiment in which the imaging element 130 positioned on the distal end 122 but does not surround the central lumen 104.

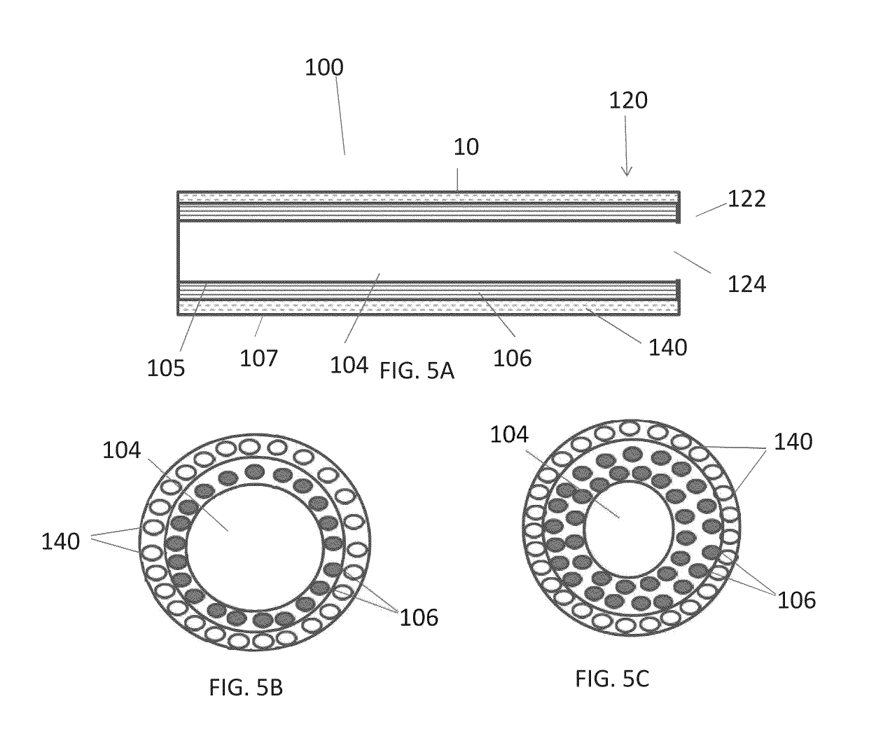

FIG. 5A depicts yet another a configuration of the catheter body 10 of the intraluminal device 100 according to certain embodiments. As shown in FIG. 5A, the catheter body 10 defines a center lumen 104. The center lumen 104 is sized to run over an implantable device (e.g. a lead connected to a pacemaker or defibrillator). The center lumen 104 terminates at an opening 124 through which a lead may be loaded into the intraluminal device 100. The catheter body 10 has an outer surface 107 and an inner lumen surface 105. The catheter body 10 terminates a distal end 122. Although the distal end 122 is shown as a flat surface, the distal end 122 may be biased at an angle.

As further shown in FIG. 5A, one or more ablation elements 106 are at least partially disposed within the catheter body. The ablation elements 106 include an active end 121 at the distal end 122 of the catheter body 110. The active end 121 is configured to release energy to ablate tissue in contact with or in close proximity to the distal end 122 of the catheter body 10. In certain embodiments, the ablation elements at least partially surround the center lumen 104. Preferably, the ablation elements 106 completely surround the center lumen. The ablation elements (e.g. optical fibers) may make up the inner surface of the catheter body, or an inner sheath may be placed between the ablation elements 106 and the center lumen 104. In certain embodiments, the one or more ablation elements 106 are optical fibers that run the length of the catheter body and terminate at the distal end 122. In this embodiment, the optical fibers are coupled to a laser system that provides laser energy through the optical fibers at the active end 121, which allows the optical fibers to ablate tissue. Alternatively, the active end 121 of the ablation elements 106 may be one or more electrodes coupled to the distal end 122. The electrodes would then be coupled to one or more signal lines that provide power to the electrodes. The ablation elements 106 are configured to ablation tissue, plaque, etc. bound to electrode lead.

In addition, the intraluminal device 100 of FIG. 5A includes at least one imaging element 140. In this embodiment, the imaging element may include one or more optical fibers surrounding the catheter body 10. As shown, the imaging optical fibers 140 surround the ablation elements 106 (which may also be optical fibers). The imaging elements 140, according to this embodiment, are preferably photoacoustic transducers formed within the optical fibers. Photoacoustic transducers are discussed in more detail hereinafter. The photoacoustic transducers may be placed at several locations along the length of the optical fiber. According to certain embodiments, at least one of the photoacoustic transducers on the optical fiber is located at or near the distal end 122 of the catheter body 10. The imaging element may obtain images of a surface before, during, or after an ablation event or while the catheter body 10 is riding along a lead. The optical fiber imaging elements 140 may partially or completely surround the catheter body 10.

FIGS. 5B and 5C provide a view of the distal end 122 of the catheter body 10 according to the embodiment depicted in FIG. 5A. As shown in both FIGS. 5B and 5C, the ablation elements are disposed around the center lumen 104 in a concentric arrangement. As shown in FIG. 5B, the ablation elements 106 are shown arranged in a single row surrounding the center lumen 104. As shown in FIG. 5C, two rows of ablation elements 106 are provided. While the ablation elements 106 are shown in rows, the ablation elements may also be dispersed in a random or densely packed fashion. In addition, the row(s) of ablation elements 106 may be equally spaced apart. Any number of rows of ablation elements 106 may be used. However, the number of rows and/or number of ablation elements 106 may be constrained by vessel size requirements of the intraluminal device 100. As further shown in FIG. 5B and FIG. 5C, the plurality of optical fiber imaging elements are also surrounding the center lumen 104.

In any of the embodiments or combinations of embodiments provided in FIGS. 2A-5C, the imaging element may include one or more radiopaque labels that are co-located with the imaging element, ablation elements, or both. When used with the external imaging modality (such as an x-ray, angiogram, etc.), the radiopaque label allows an operator to track the distal portion of the intraluminal device 100.

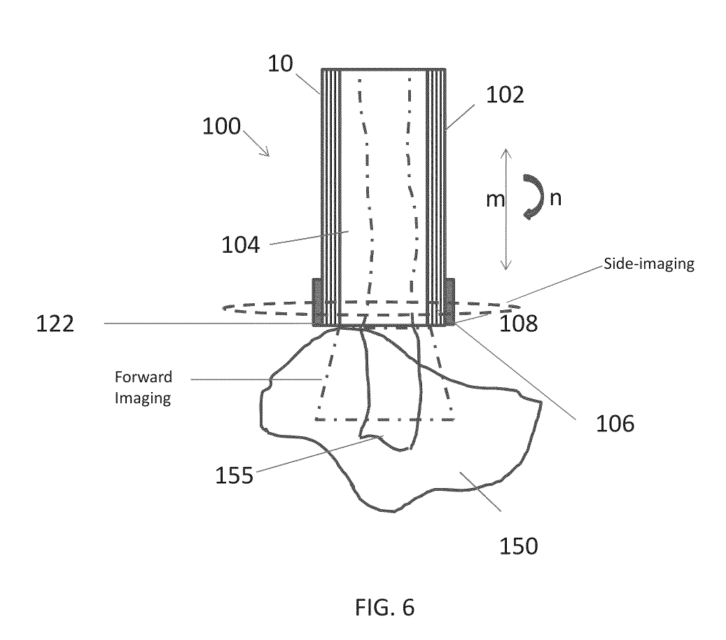

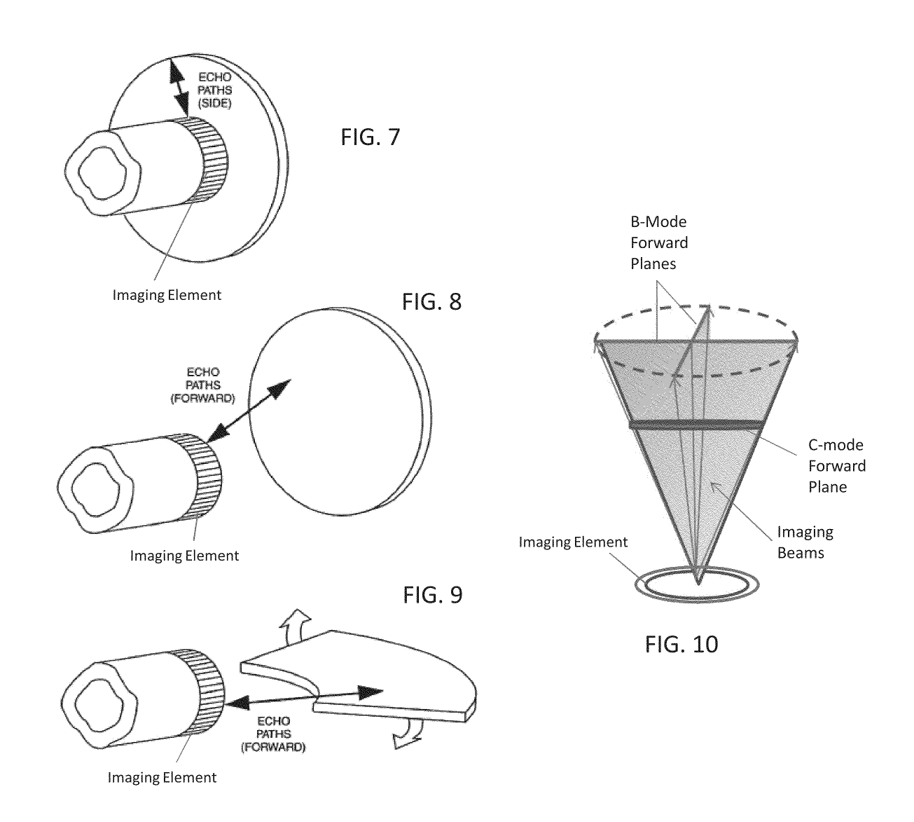

FIG. 6 is a close-up view of the lead extraction operation of the intraluminal device embodied in FIG. 2A. Specifically, FIG. 6 depicts removal of a lead as anchored within, e.g., heart tissue. It is understood that any of the embodiments depicted in FIGS. 2A-5C or combinations thereof can also be used as described hereinafter. An implanted lead 155 with its surrounding scar tissue 150 is to be removed from the body of a patient.

By way of illustration and shown in FIG. 6, the distal end 122 of the catheter body 10 with the active elements 121 of the ablation element is positioned next to the scar tissue 150. The imaging element 108 is configured to obtain images of the scar tissue 150 and the lead 155 in front of the intraluminal device 100 and to the side of the intraluminal device 100. The imaging device can obtain real-time images of the vessel lumen/tissue site/heart chamber before, during, and after an ablation procedure. Once the catheter body 10 is appropriately positioned for ablation, as determined by the obtained images, an operator may initiate ablation of the scar tissue bound to the lead 155 using the ablation elements 108. Optionally, the operator may switch back and forth between imaging and ablation, or may image during ablation. By a combination of the following actions: gradually pushing forward the catheter body 10 against the tissue mass, applying ablation energy with the ablation elements 108, and optionally rotating the catheter body 10 while ablating, the target tissue mass 150 is loosened and separated from the lead 155. This allows the lead to be removed with ease. In certain embodiments, the lead is removed by retracting the lead into the center lumen 104 of the catheter body 10.

In alternative embodiments, the devices for imaging and devices for ablating are separate. For example, a modified catheter of the catheter shown in FIGS. 2A-5C may only include the imaging element, and another modified catheter of the catheter shown in FIGS. 2A-5C may only include the ablation element. The modified imaging catheter may be positioned in a body lumen (e.g. superior vena cava or right artrium) via jugular or subclavian access to help guide the potion and cutting direction of the modified ablation catheter. In another example, the modified imaging catheter may be positioned in a body lumen (e.g. the superior vena cava or right atrium), via femoral vein, inferior vena cava, to help guide the position and cutting direction of the lead extraction catheter from a posterior position.

As discussed, the implanted structure targeted for removal is typically a cardiac lead. A cardiac lead refers to a lead that is used in connection with a heart-related device. Non-limiting examples of cardiac leads that may be removed by the inventive device include pacemaker leads, defibrillator leads, coronary sinus leads, and left ventricular pacing leads. When the device is used to remove a cardiac pacemaker lead, the distal end of the cardiac lead will normally be located within the vascular system of the patient, and in particular, within a chamber of the patient's heart (such as in an atrium or ventricle of the heart). When the implanted elongated structure is a defibrillator lead, the distal end of the structure may be located either in or about the heart of the patient. The distal ends of other types of implanted elongated structures targeted for removal may not necessarily be near the heart.

In addition to lead extraction procedures, intraluminal devices of the invention may also be used cross occluded vessels and to perform atherectomy procedures. For example, the intraluminal device can be used to ablate plaque occluding a vessel.

In certain embodiments, a lumen (such as the center lumen) of the intraluminal device may be coupled to a vacuum source. The vacuum source can be used to suction ablated material (e.g. tissue/plaque) out of a vessel during a procedure.

Although not shown in FIG. 6, the device 100 is also configured to image and ablate binding cites on portions of the lead disposed within the vessel. For example, the device 100 can ablate and image binding sites caused by plaque build-up on the elongate body of the lead as the device 100 travels on the lead to the anchor location of the lead within the heart.

Catheter bodies intended for intravascular introduction will typically have a length in the range from 50 cm to 200 cm and an outer diameter in the range from 1 French to 12 French (0.33 mm: 1 French), usually from 3 French to 9 French. In the case of coronary catheters, the length is typically in the range from 125 cm to 200 cm, the diameter is below 8 French, or below 7 French, or in the range from 2 French to 7 French. Catheter bodies of the invention can be designed to fit specifically over leads connected to implantable devices.

Catheter bodies of intraluminal devices of the invention will typically be composed of an organic polymer that is fabricated by conventional extrusion techniques. In addition, the catheter body may include an outer catheter Suitable polymers include polyvinylchloride, polyurethanes, polyesters, polytetrafluoroethylenes (PTFE), silicone rubbers, natural rubbers, and the like. In certain embodiments, catheter body includes an outer sheath. In addition, the sheath material may be metallic or include an outer metallic sheath layer. Optionally, the catheter body may be reinforced with braid, helical wires, coils, axial filaments, or the like, in order to increase rotational strength, column strength, toughness, pushability, and the like. Suitable catheter bodies may be formed by extrusion, with one or more channels being provided when desired. The catheter diameter can be modified by heat expansion and shrinkage using conventional techniques. The resulting catheters will thus be suitable for introduction to the vascular system, often the coronary arteries, by conventional techniques. Preferably, at least a portion of the catheter body is flexible.

As discussed, intraluminal devices 100 of the invention (including the embodiments depicted in FIGS. 2-5) include one or more cutting elements 106. Any cutting elements may be suitable for use in methods of the invention. The cutting elements may be an ablation element or a mechanical cutting element. Mechanical cutting elements include sharp blades or serrated surfaces that can cut through tissue or plaque. Preferably, the cutting element is a ablation element.

According to certain embodiments, the ablation element is an optical fiber coupled to a laser system. In such embodiments, the optical fiber has an active end at a distal end of the catheter body through which laser energy is emitted. The level of laser energy emitted through the optical fibers may be varied depending on the type of tissue being ablated. The use of high laser energy of more than a fluence of 60 mJ/mm2 and more than 40 Hertz has the specific goal of effectively treating heavily calcified lesions while the small tip dimension allows the high laser energy to be delivered without excessive as bubble formation. Lower laser energy levels may be used such as a fluence of 60 mJ/mm2 at 40 Hertz to ablate holes through non-calcified tissue. For calcified tissue, higher energy levels and rapid pulses of energy may be required. For example, it has been shown that rapidly emitting 2660 pulses of laser energy at a fluence of 100 mJ/mm2/80 Hertz provided fastest and best results for ablating calcified tissue, and that calcified tissue may be ablated at a minimum influence of 80 mJ/mm2/80 Hertz excimer laser parameter settings. Laser energy is transmitted from a laser system into the optical fibers. Laser systems for transmitting laser energy or other ablative energy levels into optical fibers are described in more detail in U.S. Pat. Nos. 5,188,632, 5,423,806, 5,040,5486,673,064, 5,203, 5,176,674, 5,041,108, 4,993,412, 5,032,123, and 4,834,093 along with U.S. Publication No. 2010/0152717.

In certain embodiments, the laser is an excimer laser. An excimer laser enables the disintegration of targeted tissue and can yield relatively pure disintegration without excessive thermal damage to otherwise healthy tissue. The excimer laser is a combination of argon fluoride or krypton chloride and a rare earth gas. This combination forms a laser beam having a very short wavelength and hence photons of very high energy. Excimer catheters for endovascular therapy are presently produced by the Spectranetics Corporation in the United States. Presently approved laser catheters in the United States for endovascular therapy range in diameter from 1.4 mm to 2.2 mm. The energy output, for ablating atherosclerotic tissue in coronary or peripheral arteries typically has a fluence of 60 mJ/mm2, 40 Hertz.

In other embodiments, the one or more ablation elements may include one or more electrodes. The one or more electrodes may be placed on the distal end of the catheter body. Signal lines may couple the electrodes to a power generator. The electrodes may have a variety of different shape and sizes. For example, the electrode can be a conductive plate, a conductive ring, conductive loop, or a conductive coil. The energy necessary to ablate cardiac tissue or lesion can be provided from a number of different sources including radiofrequency, microwave, ultrasound and forms of direct current (high energy, low energy and fulgutronization procedures). Radiofrequency (RF) has become the preferred source of energy for ablation procedures with electrodes.

For any ablation element used, preferably the source of energy or level of energy chosen does not disrupt the imaging of the vessel during the procedure. Alternatively, the ablation device can be interrupted or paused to allow for intermittent data acquisition. For example, imaging can occur at predetermined intervals during which laser pulses are stopped to allow for better imaging. As another example, imaging can be initiated by a doctor or technician. During this time, the laser can be deactivated to allow for better imaging. Once imaging is complete, the laser can be reactivated and pulsing can recommence (whether automatically or manually).

In addition, intraluminal devices 100 of the invention (including the embodiments depicted in FIGS. 2-5) include one or more imaging elements (108, 116, 130, and 140). Suitable imaging elements are described herein after.

Typically, the imaging element is a component of an imaging assembly. Any imaging assembly may be used with devices and methods of the invention, such as photoacoustic imaging apparatus and intravascular ultrasound (IVUS). The imaging element is used to send and receive signals to and from the imaging surface that form the imaging data.

Typically, intraluminal imaging elements image a cross-section of the vessel directly parallel to imaging element. These imaging elements are known as “side viewing” devices that produce B-mode images in a plane that is perpendicular to the longitudinal axis of the intraluminal device and passes through the imaging element. The imaging plane of B-mode side-viewing images is shown in FIG. 1. For side-viewing cross-sectional imaging, the shortened distal tips of the invention are advantageous because the shortened tip significantly reduces the distance between the cross-sectional imaging plane and distal tip of the catheter, without sacrificing protection of the imaging element. As a result, an operator can obtain images with the side-viewing imaging element right next to a blockage, in difficult tortuous angles, and in bi-furcations. Examples of side-viewing intravascular ultrasound assemblies are describe in, for example, U.S. Pat. Nos. 4,794,931, 5,000,185, 5,243,988, 5,353,798, and 5,375.602. Examples of side-viewing optical coherence tomography assemblies are described in, for example, U.S. Pat. Nos. 7,929,148, 7,577,471, and 6,546,272.

In addition, there are also “forward looking” imaging elements that image an object a distance in front of the imaging element. For example, there are devices that produce a C-mode image plane as illustrated in FIG. 8. The C-mode image plane is perpendicular to the axis of an intraluminal device and spaced in front of the imaging element. The imaging signals are transmitted at an arbitrary angle from an axis of the imaging element to image a cross-section in front of the imaging element. Other forward viewing devices produce a B-mode image in a plane that extends in a forward direction from the imaging element and parallel to the axis of the catheter. FIG. 9 exemplifies a B-Mode forward imaging plane. FIG. 10 illustrates the spatial filtering or beamformer geometry of a forward looking imaging element utilizing both B-mode and C-mode imaging.

Examples of forward-looking ultrasound assemblies are described in U.S. Pat. No. 7,736,317, 6,780,157, and 6,457,365, and in Yao Wang, Douglas N. Stephens, and Matthew O'Donnellie, “Optimizing the Beam Pattern of a Forward-Viewing Ring-Annular Ultrasoun Array for Intravascular Imaging”, Transactions on Ultrasonics, Rerroelectrics, and Frequency Control, vol. 49, no. 12, December 2002. Examples of forward-looking optical coherence tomography assemblies are described in U.S. Publication No. 2010/0220334, Fleming C. P.,Wang H., Quan, K. J., and Rollins A. M., “ Real-time monitoring of cardiac radio-frequency ablation lesion formation using an optical coherence tomography forward-imaging catheter. ,” J. Biomed. Opt. 15, (3), 030516-030513 ((2010)), and Wang H, Kang W, Carrigan T, et al; In vivo intracardiac optical coherence tomography imaging through percutaneous access: toward image-guided radio-frequency ablation. J. Biomed. Opt. 0001;16(11):110505-110505-3. doi:10.1117/1.3656966.

In certain aspects, an imaging assembly includes both side-viewing and forward-looking capabilities. These imaging assemblies utilize different frequencies that permit the imaging assembly to isolate between forward looking imaging signals and side viewing imaging signals. For example, the imaging assembly is designed so that a side imaging port is mainly sensitive to side-viewing frequencies and a forward viewing imaging port is mainly sensitive to forward viewing frequencies. Example of this type of imaging element is described in U.S. Pat. Nos. 7,736,317, 6,780,157, and 6,457,365.

In certain embodiments, the imaging element is a part of an intravascular ultrasound (IVUS) imaging assemblies. IVUS imaging assemblies produce ultrasound energy and receive echoes from which real time ultrasound images of a thin section of the blood vessel are produced. The imaging transducers of the imaging element are constructed from piezoelectric components that produce sound energy at 20-50 MHz. The image collectors of the imaging element comprise separate piezoelectric elements that receive the ultrasound energy that is reflected from the vasculature. Alternative embodiments of imaging assembly may use the same piezoelectric components to produce and receive the ultrasonic energy, for example, by using pulsed ultrasound. That is, the imaging transducer and the imaging collectors are the same. Another alternative embodiment may incorporate ultrasound absorbing materials and ultrasound lenses to increase signal to noise.

In certain embodiments, the IVUS transducer is a ring-array transducer. Phased-array IVUS catheters include a transducer array that forms a circumferential ring around the distal end of the catheter device. With the ring-array configuration, the catheter does not have to rotate to generate a cross-sectional image of a vessel. Signal processing is performed on the recorded acoustic signals to reconstruct an image (tomographic frame) whose orientation is perpendicular to the axis of the catheter body. Like rotational IVUS catheters, the phased-array catheter must be pulled back within the vessel lumen in order to provide multiple cross-sectional images across a length of the vessel. In other embodiments, the IVUS imaging assembly can be a plurality of separate transducer element placed on the distal end or distal portion of the catheter body.

IVUS data is typically gathered in segments where each segment represents an angular portion of an IVUS image. Thus, it takes a plurality of segments (or a set of IVUS data) to image an entire cross-section of a vascular object. Furthermore, multiple sets of IVUS data are typically gathered from multiple locations within a vascular object (e.g., by moving the transducer linearly through the vessel). These multiple sets of data can then be used to create a plurality of two-dimensional (2D) images or one three-dimensional (3D) image.

IVUS imaging assemblies and processing of IVUS data are described in further detail in, for example, Yock, U.S. Pat. Nos. 4,794,931, 5,000,185, and 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et at., U.S. Pat. No. 5,373,845, Seward et al., Mayo Clinic Proceedings 71(7):629-635 (1996), Packer et al., Cardiostim Conference 833 (1994), “Ultrasound Cardioscopy,” Eur. J.C.P.E. 4(2):193 (June 1994), Eberle et al., U.S. Pat. No. 5,453,575, Eberle et al., U.S. Pat. No. 5,368,037, Eberle et at., U.S. Pat. No. 5,183,048, Eberle et al., U.S. Pat. No. 5,167,233, Eberle et at., U.S. Pat. No. 4,917,097, Eberle et at., U.S. Pat. No. 5,135,486, U.S. Pub. 2009/0284332; U.S. Pub. 2009/0195514 A1; U.S. Pub. 2007/0232933; and U.S. Pub. 2005/0249391 and other references well known in the art relating to intraluminal ultrasound devices and modalities.

In yet another embodiment, the imaging element is part of an photoacoustic imaging assembly. Photoacoustic imaging assemblies include at least one imaging element to send and receive imaging signals. In one embodiment, the imaging element includes at least one acoustic-to-optical transducer. In certain embodiments, the acoustic-to-optical transducer is an Fiber Bragg Grating within an optical fiber. In addition, the imaging elements may include the optical fiber with one or more Fiber Bragg Gratings (acoustic-to-optical transducer) and one or more other transducers. The at least one other transducer may be used to generate the acoustic energy for imaging. Acoustic generating transducers can be electric-to-acoustic transducers or optical-to-acoustic transducers. The imaging elements suitable for use in devices of the invention are described in more detail below.

Fiber Bragg Gratings for imaging provides a means for measuring the interference between two paths taken by an optical beam. A partially-reflecting Fiber Bragg Grating is used to split the incident beam of light into two parts, in which one part of the beam travels along a path that is kept constant (constant path) and another part travels a path for detecting a change (change path). The paths are then combined to detect any interferences in the beam. If the paths are identical, then the two paths combine to form the original beam. If the paths are different, then the two parts will add or subtract from each other and form an interference. The Fiber Bragg Grating elements are thus able to sense a change wavelength between the constant path and the change path based on received ultrasound or acoustic energy. The detected optical signal interferences can be used to generate an image using any conventional means.

In one aspect, the imaging element utilizes the one or more Fiber Bragg Grating elements of the optical fiber in combination with an optical-to-acoustic transducer material to generate acoustic energy from optical energy. In this aspect, the acoustic-to-optical transducer (signal receiver) also acts as an optical-to-acoustic transducer (signal generator).

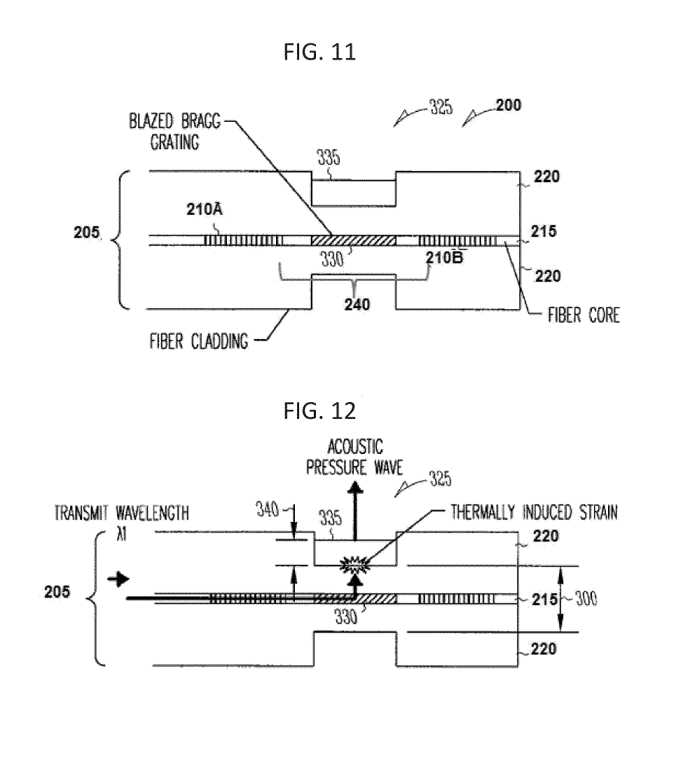

To generate the acoustic energy, imaging element may include a combination of blazed and unblazed Fiber Bragg Gratings, Unblazed Bragg Gratings typically include impressed index changes that are substantially perpendicular to the longitudinal axis of the fiber core of the optical fiber, Unblazed Bragg Gratings reflect optical energy of a specific wavelength along the longitudinal of the optical fiber. Blazed Bragg Gratings typically include obliquely impressed index changes that are at a non-perpendicular angle to the longitudinal axis of the optical fiber, Blazed Bragg Gratings reflect optical energy away from the longitudinal axis of the optical fiber, FIGS. 11 and 12 depict an imaging element according to this embodiment,

FIG. 11 shows an example of imaging element that uses Fiber Bragg Gratings to generate acoustic energy. As depicted in FIG. 11, the imaging element 200 includes an optical fiber 205 with unblazed Fiber Bragg Grating 110A and 110B and blazed Fiber Bragg Grating 330 and a photoacoustic material 335 (optical-to-acoustic transducer). The region between the unblazed Fiber Bragg Grating 210A and 210B is known as the strain sensing region 240. The strain sensing region may be, for example, 1 mm in length. The Blazed Fiber Bragg Grating 330 is implemented in the strain sensing region 240. The photoacoustic material 335 is positioned to receive the reflected optical energy from the blazed Fiber Bragg Grating 330. Although not shown, the proximal end of the optical fiber 205 is operably coupled to a laser and one or more electronic detection elements.

In operation and as depicted in FIG. 12, the blazed Fiber Bragg Grating 330 receives optical energy of a specific wavelength λ1 from a light source, e.g. a laser, and blazed Grating 330 directs that optical energy towards photoacoustic material 335. The received optical energy in the photoacoustic material 335 is converted into heat, which causes the material 335 to expand. Pulses of optical energy sent to the photoacoustic material 335 cause the photoacoustic material 335 to oscillate. The photoacoustic material 335 oscillates, due to the received optical energy, at a pace sufficient to generate an acoustic or ultrasound wave. The acoustic wave is transmitted and reflected from the imaging surface and reflected back to the imaging element. The acoustic wave reflected from the imaging surface impinges on photoacoustic transducer 335, which causes a vibration or deformation of photoacoustic transducer 335. This results in a change in length of light path within the strain sensing region 240. Light received by blazed fiber Bragg grating from photoacoustic transducer 235 and into fiber core 215 combines with light that is reflected by either fiber Bragg grating 210A or 210B (either or both may be including in various embodiments). The light from photoacoustic transducer 235 will interfere with light reflected by either fiber Bragg grating 210A or 210B and the light returning to the control unit will exhibit an interference pattern. This interference pattern encodes the ultrasonic image captured by imaging element 200. The light can be received into photodiodes within a control unit and the interference pattern thus converted into an analog electric signal. This signal can then be digitized using known digital acquisition technologies and processed, stored, or displayed as an image of the target treatment site.

The imaging element described and depicted in FIGS. 11 and 12 and other varying embodiments are described in more detail in U.S. Pat. Nos. 7,245.789, 7447,388, 7,660,492, 8,059,923 and in U.S. Patent Publication Nos. 2010/0087732 and 2012/0108943. Other exemplary optical-acoustic imaging assemblies are disclosed in more detail in U.S. Pat. Nos. 6,659,957 and 7,527,594, 7,245.789, 7447,388, 7,660,492, 8,059,923 and in U.S. Patent Publication Nos. 2008/0119739, 2010/0087732 and 2012/0108943.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. An intraluminal system for extracting an implantable device, the system comprising
a body defining a lumen and comprising a distal end, wherein the lumen is configured to receive an implantable device therein;
one or more cutting elements in an arrangement surrounding the lumen and disposed at least partially within the body; and
an imaging element located on the body.

2. The intraluminal system of claim 1, wherein the one or more cutting elements comprise ablation elements.

3. The intraluminal system of claim 2, where the ablation elements are optical fibers, and the intraluminal system is configured to deliver laser energy through the optical fibers.

4. The intraluminal system of claim 1, wherein the imaging element is located on a distal portion of the body.

5. The intraluminal system of claim 1, wherein the imaging element is configured to partially surround the distal portion of the body.

6. The intraluminal system of claim 5, wherein the imaging element is located on the distal end of the body.

7. The intraluminal system of claim 1, wherein the imaging element comprises an ultrasound transducer or a photoacoustic transducer.

8. The intraluminal system of claim 1, wherein the implantable device is a lead.

9. The intraluminal system of claim 1, wherein the one or more ablation elements are arranged in at least two rows.

10. The intraluminal system of claim 1, wherein the one or more ablation elements are arranged in a single row only.

11. The intraluminal system of claim 1, wherein the one or more ablation elements are configured to deliver laser energy from the distal end of the body.

12. An intraluminal system for extracting an implantable device, the system comprising
a body defining a first lumen and a second lumen, the first lumen configured to receive an implantable device therein;
a plurality of ablation elements in an arrangement surrounding the first and second lumens; and
an inner member moveably disposed within the second lumen of the body, wherein the inner member comprises an imaging element.

13. The intraluminal system of claim 12, wherein the imaging element is configured to obtain images of a surface when the inner member is deployed out of a distal end of the body.

14. The intraluminal system of claim 12, wherein the plurality of ablation elements comprises optical fibers.

15. The intraluminal system of claim 14, where the intraluminal system is configured to deliver laser energy through the optical fibers.

16. The intraluminal system of claim 12, wherein the imaging element is located on a distal portion of the inner member.

17. The intraluminal system of claim 16, wherein the imaging element is configured to partially surround the distal portion of the inner member.

18. The intraluminal system of claim 16, wherein the imaging element is located on a distal end of the distal portion of the inner member.

19. The intraluminal system of claim 12, wherein the imaging element comprises an ultrasound transducer or a photoacoustic transducer.

20. The intraluminal system of claim 12, wherein the implantable device is a lead.

21. The intraluminal system of claim 12, wherein the one or more ablation elements are arranged in at least two rows.

22. The intraluminal system of claim 12, wherein the one or more ablation elements are arranged in a single row only.

23. The intraluminal system of claim 12, wherein the one or more ablation elements are configured to deliver laser energy from a distal end of the body.

24. An intraluminal system comprising:
an ablation element configured to ablate tissue coupled to an implantable device; and
an imaging element configured to simultaneously image the tissue ablation.

25. The intraluminal system of claim 24, wherein the ablation element and the imaging element are located on a catheter, wherein the catheter of the ablation element and catheter of the imaging element is the same.

26. The intraluminal system of claim 24, wherein the ablation element and the imaging element are on a catheter, wherein the catheter of the ablation element is separate from the catheter of the imaging element.

27. The intraluminal system of claim 24, wherein the implantable device is a lead.

28. A method for removing an implantable device, the method comprising
ablating tissue surrounding an implantable device with an ablation element;
simultaneously imaging the tissue ablation with an imaging element.

29. The method of claim 28, wherein the ablation element and the imaging element are located on a catheter, wherein the catheter of the ablation element and catheter of the imaging element is the same.

30. The method of claim 28, wherein the ablation element and the imaging element are located on a catheter, wherein the catheter of the ablation element is separate from the catheter of the imaging element.

31. The method of claim 28, wherein the implantable device is a lead.

32. The method of claim 28, wherein the ablation element comprises one or more optical fibers.

33. The method of claim 28, wherein the imaging element comprises an ultrasound transducer or photoacoustic transducer.
